(19) United States
(12) Patent Application Publication
(10) Pub. No.: US 2019/0105253 A1
(43) Pub. Date: Apr. 11, 2019

(54) POLYACRYLATE OIL GEL COMPOSITION

(71) Applicants: Dow Global Technologies LLC, Midland, MI (US); Rohm and Haas Company, Collegeville, PA (US)

(72) Inventors: Liang Chen, Sewickley, PA (US); Yunshen Chen, Trappe, PA (US); Jennifer Koenig, Lansdale, PA (US); Xiang Qian Liu, Collegeville, PA (US); Fanwen Zeng, Audubon, PA (US)

(21) Appl. No.: 16/088,578

(22) PCT Filed: Jun. 7, 2016
(86) PCT No.: PCT/US2016/065342

§ 371 Date: Sep. 26, 2018

Related U.S. Application Data
(60) Provisional application No. 62/266,960, filed on Dec. 14, 2015.

(51) Int. Cl. A61K 8/81; A61K 8/04; A61K 8/37; A61Q 19/00

(52) U.S. Cl. CPC A61K 8/8147; A61K 8/042; A61K 2800/48; A61Q 19/00; A61K 8/37

(57) ABSTRACT

Provided are personal care compositions comprising a polyacrylate oil gel composition comprising (a) hydrophobic ester oil, and (b) one or more polymers comprising polymerized units derived from (i) 96 to 99.9 weight % of C₄-C₈ (meth)acrylate monomers, (ii) 0.1 to 2 weight % of methacrylic acid monomer, and (iii) 0 to 2 weight % of crosslinkers.

### FIELD OF THE INVENTION

This invention relates generally to polyacrylate oil gels that are useful in personal care formulations. The polyacrylate oil gels contain hydrophobic oil ester and acrylic copolymers.

### BACKGROUND

Personal care compositions contain a variety of additives that provide a wide array of benefits to the composition. One class of additives are oil thickeners that provide viscosity enhancements and impart good aesthetics, such as good sensory feel and clarity. One type of oil thickening agent known in the art are cellulose-based polymers and polyamides. These thickeners, however, come with certain drawbacks, including insufficient viscosity enhancement, high formulation temperature, and lack of consistency in viscosity control in consumer product formulations.

To this end, polyacrylate oil gels have been utilized in the art. For example, WO 2014/204937 A1 discloses personal care compositions comprising a polyacrylate oil gel containing a cosmetically acceptable hydrophobic ester oil and a polymer including at least two polymerized units. The prior art does not, however, disclose a polyacrylate oil gel according to the present invention which achieves the significant viscosity performance at low formulation temperatures while also providing a clear formulation.

Accordingly, there is a need to develop thickeners that provide significant viscosity enhancements, while not suffering from the drawbacks of the prior art.

### STATEMENT OF INVENTION

One aspect of the invention provides a polyacrylate oil gel composition comprising (a) hydrophobic ester oil, and (b) one or more polymers comprising polymerized units derived from (i) 96 to 99.9 weight % of C₄-C₈ (meth)acrylate monomers, (ii) 0.1 to 2 weight % of (meth)acrylic acid monomer, and (iii) 0 to 2 weight % of crosslinkers.

In another aspect, the invention provides a personal care composition comprising a polyacrylate oil gel comprising (a) one or more aliphatic C₈-C₂₄ alkyl triglycerides, (b) one or more polymers comprising polymerized units derived from (i) 40 to 50 weight % of butyl methacrylate, (ii) 40 to 50 weight % of ethylhexyl methacrylate, (iii) 0.1 to 2 weight % of (meth)acrylic acid monomer, and (iv) 0 to 2 weight % of a crosslinker selected from the group consisting of trimethylolpropane trimethacrylate, ethylene glycol dimethacrylate, and combinations thereof; and (c) a dermatologically acceptable carrier, wherein the polymers have an average particle size of from 105 to 140 nm.

### DETAILED DESCRIPTION

The inventors have now surprisingly found that polyacrylate oil gel compositions comprising hydrophobic ester oil and polymers having a high weight percent of polymerized units derived from C₄-C₈ (meth)acrylate and a small weight percent of methacrylic acid provide significant viscosity enhancements while retaining clarity in personal care formulations. Accordingly, the present invention provides in one aspect a polyacrylate oil gel composition comprising (a) hydrophobic oil ester, and (b) one or more polymers comprising polymerized units derived from (i) 96 to 99.9 weight % of C₄-C₈ (meth)acrylate monomers, (ii) 0.1 to 2 weight % of methacrylic acid monomer, and (iii) 0 to 2 weight % of crosslinkers.

In the present invention, “personal care” is intended to refer to cosmetic and skin care compositions for application to the skin, including, for example, body washes and cleansers, as well as leave on application to the skin, such as lotions, creams, gels, gel creams, serums, toners, wipes, liquid foundations, make-ups, tinted moisturizer, oils, face/body sprays, and topical medicines. In the present invention, “personal care” is also intended to refer to hair care compositions including, for example, shampoos, leave-on conditioners, rinse-off conditioners, styling gels, pomades, hair coloring products (e.g., two-part hair dyes), hairsprays, and mousses. Preferably, the personal care composition is cosmetically acceptable. “Cosmetically acceptable” refers to ingredients typically used in personal care compositions, and is intended to underscore that materials that are toxic when present in the amounts typically found in personal care compositions are not contemplated as part of the present disclosure. The compositions of the invention may be manufactured by processes well known in the art, for example, by means of conventional mixing, dissolving, granulating, emulsifying, encapsulating, entrapping or lyophilizing processes.

As used herein, the term “polymer” refers to a polymeric compound prepared by polymerizing monomers, whether of the same or a different type. The generic term “polymer” includes the terms “homopolymer,” “copolymer,” and “terpolymer.” As used herein, the term “polymerized units derived from” refers to polymer molecules that are synthesized according to polymerization techniques wherein a product polymer contains “polymerized units derived from” the constituent monomers which are the starting materials for the polymerization reactions. As used herein, the term “(meth)acrylate” refers to either acrylate or methacrylate, and the term “(meth)acrylic” refers to either acrylic or methacrylic. As used herein, the term “substituted” refers to having at least one attached chemical group, for example, alkyl group, alkenyl group, vinyl group, hydroxyl group, carboxylic acid group, other functional groups, and combinations thereof.

The inventive personal care compositions include one or more polymers comprising C₄-C₈ (meth)acrylate monomers. Suitable C₄-C₈ (meth)acrylate monomers include, for example, n-butyl (meth)acrylate, i-butyl (meth)acrylate, 2-ethylhexyl (meth)acrylate, cyclohexyl (meth)acrylate, n-octyl (meth)acrylate, phenyl (meth)acrylate, benzyl (meth)acrylate, and 2-phenylethyl (meth)acrylate. Preferably, the C₄-C₈ (meth)acrylate monomers comprise one or more of i-butyl methacrylate, n-butyl methacrylate, and ethylhexyl methacrylate. In certain embodiments, the polymer comprises polymerized units of C₄-C₈ (meth)acrylate monomers in an amount of from 96 to 99.9 weight %, and preferably from 98 to 99.9 weight %, based on the total weight of the polymer. In certain embodiments, the polymer comprises polymerized units derived from 40 to 90 weight % butyl (meth)acrylate monomers, and 10 to 60 weight % ethylhexyl (meth)acrylate monomers, based on the total weight of the polymer.

The polymers of the inventive personal care compositions also comprise (meth)acrylic acid monomer. In certain embodiments, the (meth)acrylic acid monomer is present in an amount of from 0.1 to 2 weight %, preferably from 0.3 to 1.5 weight %, and more preferably from 0.5 to 1 weight %, based on the total weight of the polymer.

The polymers can also include crosslinkers, such as a monomer having two or more non-conjugated ethylenically unsaturated groups, i.e., a multiethylenically unsaturated monomer. Suitable multiethylenically unsaturated monomers include, for example, di- or tri-allyl ethers and di- or tri-(meth)acrylyl esters of diols or polyols (e.g., trimethylolpropane diallyl ether, trimethylolpropane triacrylate, ethylene glycol dimethacrylate), di- or tri-allyl esters of di- or tri-acids, (e.g. diallyl phthalate), allyl (meth)acrylate, divinyl sulfone, triallyl phosphate, and divinylaromatics (e.g., divinylbenzene). In certain embodiments, the crosslinkers comprise one or more of trimethylolpropane trimethacrylate and ethylene glycol dimethacrylate. In certain embodiments, the inventive polymers comprise polymerized units of crosslinker monomers in an amount of 2 weight % or less, preferably from 0.3 to 2 weight %, and more preferably from 0.3 to 1.5 weight %, based on the total weight of the polymer.

In certain embodiments, the polymers have an average particle size of from 50 to 500 nm, preferably of from 75 to 250 nm, and more preferably of from 105 to 140 nm. Polymer molecular weights can be measured by standard methods such as, for example, size exclusion chromatography or intrinsic viscosity. In certain embodiments, the polymers of the present invention have a weight average molecular weight (M_{w}) of 10,000,000 or less, preferably 8,500,000 or less, and more preferably 7,000,000 or less as measured by gel permeation chromatography. In certain embodiments, the copolymer particles have a M_{w} of 50,000 or more, preferably 100,000 or more, and more preferably 200,000 or more, as measured by gel permeation chromatography. In certain embodiments, the polymers are present in the polyacrylate oil gel in an amount of from 0.1 to 20 weight %, preferably from 1 to 13 weight %, and more preferably from 4 to 6 weight %, based on the total weight of the polyacrylate oil gel composition.

Suitable polymerization techniques for preparing the polymers contained in the inventive personal care compositions include, for example, emulsion polymerization and solution polymerization, preferably emulsion polymerization, as disclosed in U.S. Pat. No. 6,710,161. Aqueous emulsion polymerization processes typically are conducted in an aqueous reaction mixture, which contains at least one monomer and various synthesis adjuvants, such as the free radical sources, buffers, and reductants in an aqueous reaction medium. In certain embodiments, a chain transfer agent may be used to limit molecular weight. The aqueous reaction medium is the continuous fluid phase of the aqueous reaction mixture and contains more than 50 weight % water and optionally one or more water miscible solvents, based on the weight of the aqueous reaction medium. Suitable water miscible solvents include, for example, methanol, ethanol, propanol, acetone, ethylene glycol ethyl ethers, propylene glycol propyl ethers, and diacetone alcohol. In certain embodiments, the aqueous reaction medium contains more than 90 weight % water, preferably more than 95 weight % water, and more preferably more than 98 weight % water, based on the weight of the aqueous reaction medium.

The polymers of the present invention may be isolated by a spray drying process. While spray drying is one preferred embodiment of how to produce the dry powder, other suitable methods include, for example, freeze drying, a two-step process including the steps of (i) pan drying the emulsion and then (ii) grinding the pan dried material into a fine powder, coagulation of the acrylic emulsion and collection of the powder by filtration followed by washing and drying, fluid bed drying, roll drying, and freeze drying. Suitable techniques for spray drying the polymer beads of the present invention are known in the art, for example, as described in US 2014/0113992 A1. In certain embodiments, anti-caking agents are used when spray drying the polymer beads. Suitable anti-caking agents include, for example, mineral fillers (e.g., calcium carbonate, kaolin, titanium oxide, talc, hydrated alumina, bentonite, and silica), solid polymer particles with a T_{g} or Tₘ greater than 60° C. (e.g., polymethylmethacrylate, polystyrene, and high density polyethylene), and water soluble polymers with a T_{g} greater than 60° C. (e.g., polyvinyl alcohol and methylcellulose). The anti-caking agent can be mixed in the acrylic suspension prior to spray drying or introduced as a dry powder in the spray drying process. In certain embodiments, the anti-caking agent coats the polymer beads to prevent the beads from sticking to each other inner wall of the dryer. In certain embodiments, the anti-caking agent is present in an amount of from 0 to 20 weight %, and more preferably from 0.01 to 10 weight %, based on the total weight of the polymer beads.

The polyacrylate oil gel compositions of the present invention also contain a cosmetically acceptable hydrophobic ester oil. In general, any hydrophobic ester oil or mixtures thereof which are toxicologically safe for human or animal use may constitute the oil base of the present invention. In certain embodiments, the hydrophobic ester oil comprises aliphatic C₈-C₂₄ alkyl triglycerides. Suitable hydrophobic ester oils include, for example, caprylic/capric triglycerides, saturated fatty esters and diesters (e.g., isopropyl palmitate, octyl palmitate, butyl stearate, isocetyl stearate, octadodecyl stearate, octadodecyl stearoyl stearate, diisopropyl adipate, and dioctyl sebacate), and animal oils and vegetable oils (e.g., mink oil, coconut oil, soybean oil, palm oil, corn oil, cocoa butter, sesame oil, sunflower seed oil, jojoba oil, olive oil, and lanolin oil). In certain embodiments, the hydrophobic ester oil is diffused in an oil base. Suitable oil bases include any oil or mixture of oils which are conventionally used in personal care products including, for example, paraffin oils, paraffin waxes, and fatty alcohols (e.g., stearyl alcohol, isostearyl alcohol, and isocetyl alcohol). In certain preferred embodiments, the hydrophobic ester oil comprises one or more of caprylic/capric triglycerides and sunflower seed oil. In certain embodiments, the hydrophobic ester oils are present in the polyacrylate oil gel in an amount of from 80 to 99.9 weight %, preferably from 87 to 99 weight %, and more preferably from 94 to 96 weight %, based on the total weight of the polyacrylate oil gel composition.

Polyacrylate oil gels according to the present invention may be formulated by conventional mixing processes known to those skilled in the art. In certain embodiments, the formulation temperature is from 25° C. to 150° C., preferably from 50° C. to 100° C., and more preferably from 60° C. to 80° C. In certain embodiments, the inventive personal care composition includes the polyacrylate oil gel described herein in an amount of at least 0.5 weight %, at least 2 weight %, or at least 4 weight %, by weight of the composition. In certain embodiments, the inventive skin care compositions comprise the particles described herein in an amount of no more than 25 weight %, no more than 30 weight %, or no more than 40 weight %, by weight of the composition.

The inventive personal care compositions also include a dermatologically acceptable carrier. Such material is typically characterized as a carrier or a diluent that does not cause significant irritation to the skin and does not negate the activity and properties of active agent(s) in the composition. Examples of dermatologically acceptable carriers that are useful in the invention include, without limitation, water, such as deionized or distilled water, emulsions, such as oil-in-water or water-in-oil emulsions, alcohols, such as ethanol, isopropanol or the like, glycols, such as propylene glycol, glycerin or the like, creams, aqueous solutions, oils, ointments, pastes, gels, lotions, milks, foams, suspensions, powders, or mixtures thereof. The aqueous solutions may contain cosolvents, e.g., water miscible cosolvents. Suitable water miscible cosolvents include, for example, ethanol, propanol, acetone, ethylene glycol ethyl ethers, propylene glycol propyl ethers, and diacetone alcohol. In some embodiments, the composition contains from about 99.99 to about 50 percent by weight of the dermatologically acceptable carrier, based on the total weight of the composition.

Other additives may be included in the compositions of the invention such as, but not limited to, abrasives, absorbents, aesthetic components such as fragrances, pigments, colorings/colorants, essential oils, skin sensates, astringents (e.g., clove oil, menthol, camphor, eucalyptus oil, eugenol, menthyl lactate, witch hazel distillate), preservatives, anti-caking agents, a foam building agent, antifoaming agents, antimicrobial agents (e.g., iodopropyl butylcarbamate), antioxidants, binders, biological additives, buffering agents, bulking agents, chelating agents, chemical additives, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents (e.g., hydroquinone, kojic acid, ascorbic acid, magnesium ascorbyl phosphate, ascorbyl glucosamine), skin-conditioning agents (e.g., humectants, including miscellaneous and occlusive), skin soothing and/or healing agents (e.g., panthenol and derivatives (e.g., ethyl panthenol), aloe vera, pantothenic acid and its derivatives, allantoin, bisabolol, and dipotassium glycyrrhizinate), skin treating agents, vitamins (e.g., Vitamin C) and derivatives thereof, silicones, and fatty alcohols. The amount of option ingredients effective for achieving the desired property provided by such ingredients can be readily determined by one skilled in the art.

Some embodiments of the invention will now be described in detail in the following Examples.

### EXAMPLES

### Example 1

### Preparation of Exemplary Polymer and Comparative Polymers

Exemplary polymers in accordance with the present invention and comparative polymers contain the components recited in Table 1.

TABLE 1
Exemplary and Comparative Polymers Particles

| Sample | Monomer (wt %) |
|---|---|
| NP1 | 49.75 iBMA/49.75 EHMA/0.5 MAA |
| NP2 | 49.5 iBMA/49.5 EHMA/1.0 MAA |
| NP3 | 49.25 iBMA/49.25 EHMA/1.5 MAA |
| NC1* | 48.5 iBMA/48.5EHMA/3 MAA |
| NC2* | 47.5 iBMA/47.5 EHMA/5 MAA |
| XP1 | 49.5 iBMA/49.5 EHMA/1.0 MAA//0.3 TMPTMA |
| XP2 | 49.5 iBMA/49.5 EHMA/1.0 MAA//0.3 EGDMA |
| XP3 | 49.5 iBMA/49.5 EHMA/1.0 MAA//0.46 TMPTMA |
| XP4 | 49.5 iBMA/49.5 EHMA/1.0 MAA//0.6 TMPTMA |
| XP5 | 49.5 iBMA/49.5 EHMA/1.0 MAA//0.6 EGDMA |
| XP6 | 49.5 iBMA/49.5 EHMA/1.0 MAA//0.9 TMPTMA |
| XP7 | 49.5 iBMA/49.5 EHMA/1.0 MAA//0.9 EGDMA |
| XP8 | 49.5 iBMA/49.5 EHMA/1.0 MAA//1.2 TMPTMA |
| XP9 | 49.5 iBMA/49.5 EHMA/1.0 MAA//1.2 EGDMA |
| XP10 | 49.5 iBMA/49.5 EHMA/1.0 MAA//1.5 EGDMA |
| XP11 | 79.5 iBMA/18.5 EHMA/1.0 MAA//0.45 TMPTMA |
| XP12 | 99 iBMA/1.0 MAA//0.45 TMPTMA |
| XC1* | 49.5 iBMA/49.5 EHMA/1.0 MAA//3 TMPTMA |
| XC2* | 49.5 iBMA/49.5 EHMA/1.0 MAA//3 EGDMA |

iBMA = isobutyl methacrylate
EHMA = ethylhexyl methacrylate
MAA = methacrylic acid
TMPTMA = trimethylolpropane trimethacrylate
EGDMA = ethylene glycol dimethacrylate
*Comparative

Synthesis of exemplary polymer XP8 was carried out as follows. A three liter round bottom flask was equipped with a mechanical overhead stirrer, heating mantle, thermocouple, condenser and inlets for the addition of monomer, initiator and nitrogen. The kettle was charged with 470 grams deionized water and 7.46 grams of DS-4 (Polystep A-16-22: sodium dodecylbenzene sulfonate from Stepan). The kettle contents were set to stir with a nitrogen flow and heated to 87-89° C. To a plastic lined vessel, 7 grams of DS-4 and 181.65 grams deionized water was added and mixed with overhead stirring. 277.2 grams of Isobutyl Methacrylate, 277.2 grams of 2-Ethylhexyl Methacrylate, 5.6 grams of Methacrylic Acid and 6.72 grams of Trimethylolpropane Trimethacrylate was charged to the vessel and allowed to form a smooth, stable monomer emulsion. An initial catalyst charge of 0.28 grams of ammonium persulfate and 12.71 grams of deionized water was prepared and set aside. A kettle buffer solution of 1.92 grams of ammonium bicarbonate and 12.71 grams of deionized water was prepared and set aside. A preform seed of 22.38 grams was removed from the stable monomer emulsion and put into a small beaker. A rinse of 16.8 grams of deionized water was prepared. A co-feed catalyst charge of 0.28 grams of ammonium persulfate and 49.22 grams of deionized water was prepared and set aside.

When the kettle was at temperature, the kettle buffer solution and initial catalyst solution were added to the reactor, followed by the perform seed and rinse. The reaction was monitored for a small exotherm. After the exotherm, the temperature control was adjusted to 83-85° C. The monomer emulsion feed was added to the kettle, sub-surface, at a rate of 4.38 grams/minute for 15 minutes. After 15 minutes, the rate was increased to 8.77 grams/minutes for 75 minutes, giving a total feed time of 90 minutes. While the monomer emulsion feed was added to the kettle, the co-feed catalyst solution was also added over 90 minutes at a rate of 0.55 grams/minute. At the completion of the feeds, 16.8 grams of deionized water was added as a rinse. The reaction was then held for 20 minutes at 83-85° C.

During the hold, a chase promoter of 3.77 grams of a 0.15% iron sulfate heptahydrate solution was prepared. A chase activator solution of 1.12 grams of isoascorbic acid dissolved in 36.40 grams of deionized water was prepared. A chase catalyst solution of 2.14 grams of 70% tert-butyl hydroperoxide in 35.40 grams of deionized water was prepared.

At 80° C., the chase promoter solution was added as a shot to the kettle. The kettle contents were then cooled to 70° C., while adding the chase activator and chase catalyst solutions separately by syringe over 60 minutes at a feed rate of 0.7 grams/minute. The reaction was held for 10 minutes, and then cooled to room temperature. At room temperature, the emulsion was filtered through a 100 mesh bag.

Exemplary polymers NP1-NP3, XP1-XP7, and XP9-CP12, and comparative polymers NC1, NC2, XC1, and CX2 were prepared substantially as described above, with the appropriate changes in monomer and monomer amounts as recited in Table 1.

### Example 2

### Particle Size Characterization of Exemplary Polymers

Exemplary and comparative polymers as prepared in Example 1 were evaluated for particle size as shown in Table 2.

TABLE 2
Particle Size Characterization

| Sample | Particle Size (nm) |
|---|---|
| NP1 | 126 |
| NP2 | 123 |
| NP3 | 122 |
| XP3 | 120 |
| XP4 | 122 |

The particle size distributions of exemplary polymers was determined by light diffraction using a Malvern Mastersizer 2000 Analyzer equipped with a 2000 uP module. Approximately 0.5 g of polymer emulsion samples were pre-diluted into 5 mL of 0.2 weight % active Triton 405 in degassed, DI water (diluents). The pre-diluted sample was added drop-wise to the diluent filled 2000 uP module while the module was pumped at 1100 rpm. Red light obscurations were targeted to be between 4 and 8%. Samples were analyzed using a Mie scattering module (particle real refractive index of 1.48 and absorption of zerp: Diluent real refractive index of 1.330 with absorption of zero). A general purpose (spherical) analysis model with “normal sensitivity” was used to analyze the diffraction patterns and convert them into particle size distributions.

### Example 3

### Spray Drying of Exemplary and Comparative Polymers

Exemplary and comparative polymers as prepared in Example 1 were spray dried according to the following procedure. A two-fluid nozzle atomizer was equipped on a Mobile Minor spray dryer (GEA Process Engineering Inc.). The spray drying experiments were performed under an inert atmosphere of nitrogen. The nitrogen supplied to the atomizer at ambient temperature was set at 1 bar and 50% flow, which is equivalent to 6.0 kg/hour of flow rate. The polymer emulsion was fed into the atomizer at about 30 mL/min using a peristaltic pump (Masterflex L/S). Heated nitrogen was used to evaporate the water. The inlet temperature was set at 140° C., and the outlet temperature was equilibrated at 40-50° C. by fine tuning the emulsion feed rate. The resulting polymer powder was collected in a glass jar attached to the cyclone and subsequently vacuum dried at room temperature to removed residual moisture.

### Example 4

Viscosity of Polyacrylate Oil Gel Prepared from Spray Dried Exemplary and Comparative Polymers

The viscosities of exemplary polyacrylate oil gels formed from exemplary and comparative polymers as prepared in Example 1 and spray dried according to the procedure in Example 3 are shown in Table 3.

TABLE 3
Viscosities of Polyacrylate Oil Gel from Spray Dried Acrylic Polymer

| | | Polymer | | |
|---|---|---|---|---|
| | | Concentration | | |
| Sample | Oil⁺ | in Oil (wt %) | Viscosity (cP) | Clarity |
| NP1 | SSO | 6 | 3,000* | clear |
| NP2 | SSO | 6 | 8,000* | clear |
| NP3 | SSO | 6 | 167,000* | clear |
| NC1 | SSO | 6 | Unstable oil gel | hazy |
| NC2 | SSO | 6 | Unstable oil gel | hazy |
| XP1 | SSO | 4 | 1,700* | clear |
| XP2 | SSO | 4 | 8,000* | clear |
| XP3 | SSO | 4 | 6,500* | clear |
| XP4 | SSO | 4 | 3,900* | clear |
| | SSO | 6 | 13,000* | clear |
| XP5 | SSO | 4 | 10,000* | clear |
| | SSO | 6 | 10,000* | clear |
| XP6 | SSO | 4 | 15,700* | slightly |
| | | | | translucent |
| XP7 | SSO | 4 | 10,000* | slightly |
| | | | | translucent |
| | SSO | 6 | 30,000* | slightly |
| | | | | translucent |
| XP8 | SSO | 4 | 8,600* | translucent |
| XP9 | SSO | 4 | 9,800* | translucent |
| XP10 | SSO | 4 | 8,600* | translucent |
| XP11 | CCT | 6 | 600** | translucent |
| XP12 | CCT | 6 | 300** | translucent |
| XC1 | SSO | 4 | Unstable oil gel | hazy |
| XC2 | SSO | 4 | Unstable oil gel | hazy |

⁺CCT = Caprylic/capric triglyceride is available from Rita Corporation;
SSO = Sunflower seed oil available from Spectrum.
*Measured with Brookfield viscometer, Spindle S96 at 6 rpm
**Measured with Brookfield viscometer, Spindle S93 at 30 rpm

Exemplary polyacrylate oil gels as evaluated in Table 4 above were formulated by heating the cosmetic oil and polymer to 70° C. under stirring (EuroStar 60, IKA) at 500 rpm for 1 hour. The mixture was then cooled to room temperature.

The results demonstrate that the inventive polyacrylate oil gels exhibit far superior viscosity enhancement and clarity when compared with comparative oil gels prepared from comparative polymers.

What is claimed is:

1. A polyacrylate oil gel composition comprising:
(a) hydrophobic ester oil; and
(b) one or more polymers comprising polymerized units derived from
(i) 96 to 99.9 weight % of C₄-C₈ (meth)acrylate monomers,
(ii) 0.1 to 2 weight % of (meth)acrylic acid monomer, and
(iii) 0 to 2 weight % of crosslinkers.

2. The composition of claim 1, wherein the C₄-C₈ (meth)acrylate monomers are selected from the group consisting of ethylhexyl (meth)acrylate, butyl (meth)acrylate, and combinations thereof.

3. The composition of claim 1, wherein one or more polymers comprise polymerized units derived from 40 to 90 weight % butyl (meth)acrylate monomers, and 10 to 60 weight % ethylhexyl (meth)acrylate monomers.

4. The composition of claim 1, wherein the polymers comprise 0.3 to 2 weight % polymerized units derived from crosslinkers.

5. The composition of claim 4, wherein the crosslinkers comprise one or more of trimethylolpropane trimethacrylate and ethylene glycol dimethacrylate.

6. The composition of 5, wherein the crosslinkers comprise trimethylolpropane trimethacrylate.

7. The composition of claim 5, wherein the crosslinkers comprise ethylene glycol dimethacrylate.

8. The composition of claim 1, wherein the hydrophobic ester oil comprises one or more aliphatic C₈-C₂₄ alkyl triglycerides.

9. The composition of claim 1, wherein the polymers have an average particle size of from 50 to 500 nm.

10. A personal care composition comprising a polyacrylate oil gel comprising:
(a) one or more aliphatic C₈-C₂₄ alkyl triglycerides;
(b) one or more polymers comprising polymerized units derived from
(i) 40 to 50 weight % of butyl methacrylate,
(ii) 40 to 50 weight % of ethylhexyl methacrylate,
(iii) 0.1 to 2 weight % of (meth)acrylic acid monomer, and
(iv) 0 to 2 weight % of a crosslinker selected from the group consisting of trimethylolpropane trimethacrylate, ethylene glycol dimethacrylate, and combinations thereof; and
(c) a dermatologically acceptable carrier,
wherein the polymers have an average particle size of from 105 to 140 nm.
